(19) United States
(12) Patent Application Publication
(10) Pub. No.: US 2026/0028395 A1
(43) Pub. Date: Jan. 29, 2026

(54) TREATMENT OF OPHTHALMOLOGIC DISEASES

(71) Applicant: Genentech, Inc., South San Francisco, CA (US)

(72) Inventor: Jeffrey R. WILLIS, San Francisco, CA (US)

(73) Assignee: Genentech, Inc., South San Francisco, CA (US)

(21) Appl. No.: 19/279,391

(22) Filed: Jul. 24, 2025

(30) Foreign Application Priority Data
Jul. 26, 2024 (WO) PCT/US2024/039807

(51) Int. Cl. C07K 16/22; A61K 39/00; A61P 27/02

(52) U.S. Cl. CPC C07K 16/22; A61P 27/02; A61K 2039/505; A61K 2039/545; C07K 2317/31

(57) ABSTRACT

The current invention relates to antibodies, which bind to human vascular endothelial growth factor (VEGF) and human angiopoietin-2 (ANG2) for use in the treatment of ocular vascular diseases. Specifically, the invention relates to a method of preventing or reducing epiretinal membrane (ERM) formation in an eye of a patient by administering a bispecific antibody which binds to VEGF and to ANG-2.

### CROSS-REFERENCE TO RELATED APPLICATIONS

This application claims the benefit of priority to International Application Number, PCT/US2024/039807 filed Jul. 26, 2024, of which is incorporated herein by reference in its entirety.

### SEQUENCE LISTING

This application contains a Sequence Listing which has been submitted electronically in XML format and is hereby incorporated by reference in its entirety. Said XML copy, created on Jul. 15, 2025, is named P39451-US-1_SEQ_List.xml and is 25,834 bytes in size

### FIELD OF THE DISCLOSURE

The current invention relates to antibodies that bind to human vascular endothelial growth factor (VEGF) and to human angiopoietin-2 (ANG-2) for use in the treatment of ocular vascular diseases.

### BACKGROUND OF THE DISCLOSURE

Ocular vascular diseases such as diabetic retinopathy in particular diabetic macular edema (DME) are severe diseases leading to often to visual loss and blindness. DME, a complication of diabetic retinopathy (DR), can develop at any stage of the underlying disease of retinal microvasculature. DME occurs with increasing frequency as the underlying DR worsens from non-proliferative DR (NPDR) to proliferative DR (PDR). DME is the most common cause of moderate and severe visual impairment in patients with DR, and if left untreated can lead to a loss of 10 or more letters in visual acuity (VA) within 2 years in approximately 50% of patients. DME affects approximately 14% of patients with diabetes and can be found in patients with both Type 1 and Type 2 diabetes. In 2013, the worldwide population of people with diabetes was approximately 382 million, and it is estimated to grow to 592 million by 2035 (International Diabetes Federation 2013).

With advances in imaging technology, DME is now often diagnosed by optical coherence tomography (OCT) rather than the traditional Early Treatment Diabetic Retinopathy Study (ETDRS) ophthalmoscopy-based criteria. On a molecular level, DME is a result of a vascular endothelial growth factor-A (VEGF-A)-mediated increase in vessel permeability and loss of pericytes, consequent to hypoxia-mediated release of pro-angiogenic, hyperpermeability, and pro-inflammatory mediators. VEGF also upregulates a homeostatic factor, angiopoietin-2 (Ang-2), which acts as an antagonist of the Tie2 receptor tyrosine kinase on endothelial cells, counteracting vessel stabilization maintained through Ang-1-dependent Tie2 activation. Therefore, Ang-2 acts as a vascular destabilization factor, rendering the vasculature more elastic and amenable to endothelial barrier breakdown and sprouting. The excess of Ang-2 and VEGF in the retinal tissues promotes vessel destabilization, vascular leakage, and neovascularization. Ang-2 is also involved in inflammatory pathways such as lymphocyte recruitment. In summary, both VEGF-A and Ang-2 are recognized as key factors mediating diabetic eye disease pathogenesis.

Although macular laser used to be the standard of care (SOC) for treatment of DME, the development of anti-VEGF pharmacotherapy in the past 10 years has led to dramatic improvements in visual outcomes for patients with DME. Other available approved options for the treatment of DME include periocular or intravitreal (IVT) steroids and steroid implants.

Despite the strong efficacy achieved with anti-VEGF therapies in DME, a significant proportion of patients do not experience clinically meaningful improvements in vision in the real world. Frequent IVT administration is required to achieve, and in some cases, to maintain the observed early benefits of DME treatment over a long period of time. The current SOC for administration of anti-VEGF injections requires patients to undergo frequent clinical examinations and IVT injections. This imposes a significant burden on patients, caregivers, treating physicians, and the healthcare system.

In order to better address the complex nature of DME and improve long-term outcomes, attempts have been made to target more than one pathway involved in its pathogenesis. Faricimab (Vabysmo, F. Hoffmann-La Roche), a bispecific antibody that blocks both Angiopoietin-2 (Ang-2) and VEGF-A, is one of these molecules.

The Ang/Tie pathway is a key player in the development and homeostasis of vessels. Activation of Tie2 by Ang-1 leads to vascular stability. Ang-2 on the other hand acts predominantly as an antagonist of Ang-1. When Ang-2 is upregulated, as is the case in multiple retinal pathologies including diabetic retinopathy, it destabilizes the vasculature and enhances the vessels' sensitivity to VEGF-A. Preclinical studies have shown that Ang-2 and VEGF-A act in synergy to drive vascular leakage, neovascularization and inflammation, making combined inhibition of Ang-2 and VEGF-A a potentially valuable approach to improve vascular stability, and as a result disease severity and long-term outcomes.

Epiretinal membranes (ERMs) or preretinal fibrosis are common findings among patients with DME, occurring in about 14% of eyes with center-involving disease and 9.5% of eyes treated with intravitreal injections for 2 years. Symptomatic ERMs can lead to decreased visual acuity, metamorphopsia, and potentially require surgery.

There remains a need to reduce the incidence of ERMs in DME and improve severity and progression in DME.

### SUMMARY OF THE DISCLOSURE

The etiology of ERMs is likely multifactorial, with a common phenotype of preretinal fibrotic tissue and a potential association with underlying inflammation and poor retinal vasculature control. On a pathophysiological level, ERM tissue from eyes with ischemic retinal diseases tend to have increased Ang-2 and Tie2 expression. Furthermore, preclinical studies show that Ang-2 promotes inflammation, vascular permeability, and fibrosis. Under normal conditions, Ang-1 binds to the Tie2 receptor to activate a cascade of events that stabilizes the vasculature and maintains blood vessels in a quiescent state. In disease states when Ang-2 levels surpass Ang-1, Ang-2 occupies the Tie2 receptor but does not activate it, thereby enhancing VEGF activity driving excessive permeability, inflammation, pathological angiogenesis, and fibrosis. Without being bound by a theory, it is suggested that the incidence of ERMs in DME might be reduced through Ang-2 blockade.

One aspect of the disclosure provides a method of preventing or reducing epiretinal membrane (ERM) formation in an eye of a patient. Such method includes administering to the patient an effective amount of a bispecific antibody which binds to human vascular endothelial growth factor (VEGF) and to human angiopoietin-2 (ANG-2). In certain embodiments, such patient suffers from diabetic macular edema (DME).

Another aspect of the disclosure provides a bispecific antibody which binds to human VEGF and to human ANG-2 for use in method of preventing or reducing ERM formation in an eye of a patient. In certain embodiments, such patient suffers from DME.

Another aspect of the disclosure provides a formulation comprising a bispecific antibody which binds to human VEGF and to human ANG-2 for use in method of preventing or reducing ERM formation in an eye of a patient. In certain embodiments, such patient suffers from DME.

Another aspect of the disclosure provides a method of treating a patient suffering from DME. Such method includes administering to the patient an effective amount of a bispecific antibody which binds to human VEGF and to human ANG-2; and measuring ERM in an eye of the patient after 16 and/or 48 weeks of treatment.

Yet another aspect of the disclosure provides a method of treating a patient suffering from DME. Such method includes administering to the patient an effective amount of a bispecific antibody which binds to human VEGF and to human ANG-2; measuring ERM in an eye of the patient after 16 and/or 48 weeks of treatment; and adjusting administration dosing interval based on the ERM presence.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1 is a patient flow diagram showing analysis of ERM formation in the Phase 3 YOSEMITE and RHINE Trials. BL, baseline; Q8W=every 8 weeks; T&E, treat and extend-based regimen; w/, with; w/o, without.

FIG. 2 shows cumulative proportion of eyes without ERMs at baseline that developed ERMs after 1 and 2 Years (Weeks 52 and 100) (ERM Analysis Population). Missing data were not imputed, and eyes with no postbaseline ERM results were excluded from the analysis. ERMs are defined as the presence of significant distortion of macular architecture in the central subfield. ^{a} The denominator is the number of eyes with no ERMs at baseline who had ERM status available through the study. Once an eye was noted as having an ERM, it was accounted for in the numerator with the assumption that ERMs remain present. ^{b} The adjusted OR and 95% CI were produced using a multivariate logistic regression model including treatment group, baseline BCVA score (<64EDTRS letters [<20/50 Snellen] vs. ≥64 letters [≥20/50 Snellen]), prior IVT anti-VEGF therapy (yes vs no), region (US and Canada, and the rest of the world), and study (YOSEMITE vs RHINE) as covariates using cumulative data through week 100. Risk refers to the odds from logistic regression. The P values are nominal and are not adjusted for multiplicity; no formal statistical conclusion should be made based on the P values. CI, confidence interval; ITT, intent-to-treat; OR, odds ratio.

FIG. 3 shows a) BCVA, b) CST, c) proportion of eyes with intraretinal fluid (IRF) and subretinal fluid (SRF), and d) macular leakage at baseline and year 2 (Week 96 or 100) in eyes with and without ERM formation (ERM analysis population). ERM status was derived using postbaseline ERM assessments. Missing data were not imputed, and patients with invalid BCVA/no CST/no IRF/SRF values or no postbaseline ERM results were excluded from the analysis. The bars represent 95% CIs for pooled data. No hypothesis testing was done, thus no p values were derived. Baseline was defined as the last available measurement obtained on or prior to randomization. Central subfield thickness (CST) was defined as the distance between the internal limiting membrane and Bruch's membrane in the central 1-mm diameter of the ETDRS grid, as assessed by Central Reading Center (CRC) assessors. n, number of eyes at that visit; RF, retinal fluid.

FIG. 4 shows proportion of eyes in the faricimab T&E arm on each treatment interval at year 2 (week 96) among eyes with and without ERM formation during the study (ERM analysis population).

FIG. 5 shows examples of optical coherence tomography (OCT) scans for eyes a) with no ERMs causing significant distortion of the macular architecture at baseline and b) with ERM formation at week 48.

### DETAILED DESCRIPTION OF THE DISCLOSURE

As provided above, one aspect of the disclosure provides a method of preventing or reducing epiretinal membrane (ERM) formation in an eye of a patient. Such method includes administering to the patient an effective amount of a bispecific antibody which binds to human VEGF and to human ANG-2.

In certain embodiments, the patient suffers from DME.

Another aspect of the disclosure provides a bispecific antibody which binds to human VEGF and to human ANG-2 for use in method of preventing or reducing ERM formation in an eye of a patient. In certain embodiments, such patient suffers from DME.

Another aspect of the disclosure provides a formulation comprising a bispecific antibody which binds to human VEGF and to human ANG-2 for use in method of preventing or reducing ERM formation in an eye of a patient. In certain embodiments, such patient suffers from DME.

Another aspect of the disclosure provides a method of treating a patient suffering from DME. Such method includes administering to the patient an effective amount of a bispecific antibody which binds to human VEGF and to human ANG-2; and measuring ERM in an eye of the patient after 16 and/or 48 weeks of treatment.

Yet another aspect of the disclosure provides a method of treating a patient suffering from DME. Such method includes administering to the patient an effective amount of a bispecific antibody which binds to human VEGF and to human ANG-2; measuring ERM in an eye of the patient after 16 and/or 48 weeks of treatment; and adjusting administration dosing interval based on the ERM presence.

In certain embodiments of the methods or bispecific antibodies (for use), medicaments or pharmaceutical formulations of the disclosure, the effective amount of the bispecific antibody is sufficient to reduce ERM formation after 48 weeks of treatment. In certain embodiments, the effective amount of the bispecific antibody is sufficient to reduce ERM formation after 96 weeks of treatment.

In certain embodiments of the methods or bispecific antibodies (for use), medicaments or pharmaceutical formulations of the disclosure, the ERM formation, if present, is less than 0.5 relative to the ERM formation with standard of care treatment (such as aflibercept).

In certain embodiments of the methods or bispecific antibodies (for use), medicaments or pharmaceutical formulations of the disclosure, the ERM formation, if present, is less than 0.4 relative to the ERM formation without any treatment.

“Diabetic Macular Edema” (DME), as used herein, refers to a serious eye condition that affects people with diabetes (type 1 or 2). Macular edema occurs when blood vessels in the retina leak into the macula and fluid and protein deposits collect on or under the macula of the eye and causes it to thicken and swell (edema). The swelling may distort a person's central vision, as the macula is near the center of the retina at the back of the eyeball. The primary symptoms of DME include, but are not limited to, blurry vision, floaters, loss of contrast, double vision, and eventual loss of vision. The pathology of DME is characterized by breakdown of inner the blood-retinal barrier, normally preventing fluid movement in the retina, thus allowing fluid to accumulate in the retinal tissue, and presence of retinal thickening. DME is presently diagnosed during an eye examination consisting of a visual acuity test, which determines the smallest letters a person can read on a standardized chart, a dilated eye exam to check for signs of the disease, imaging tests such as optical coherence tomography (OCT) or fluorescein angiography (FA) and tonometry, an instrument that measures pressure inside the eye. The following studies are also performed to determine treatment: optical coherence tomography (OCT), fluorescein angiography, and color stereo fundus photography. DME can be broadly characterized into two main categories—Focal and Diffuse. Focal DME is characterized by specific areas of separate and distinct leakage in the macula with sufficient macular blood flow. Diffuse DME results from leakage of the entire capillary bed surrounding the macula, resulting from a breakdown of the inner blood-retina barrier of the eye. In addition to Focal and Diffuse, DME is also categorized based on clinical exam findings into clinically significant macular edema (CSME), non-CSME and CSME with central involvement (CSME-CI), which involves the fovea. The present invention includes methods to treat the above-mentioned categories of DME.

As used herein, the term “a patient suffering from” may include a subset of population which is more susceptible to DME or AMD or may show an elevated level of a DME-associated or an AMD-associated biomarker. For example, “a subject in need thereof” may include a subject suffering from diabetes for more than 10 years, have frequent high blood sugar levels or high fasting blood glucose levels. In certain embodiments, the term “a patient suffering from” includes a subject who, prior to or at the time of administration of the bispecific anti-VEGF/ANG2 antibody, has or is diagnosed with diabetes. In certain embodiments, the term “a patient suffering from” includes a subject who, prior to or at the time of administration of the anti-VEGF/ANG2 antibody, is more than 50 years old. In some embodiments, the term “a patient suffering from” includes subjects who are smokers, or subjects with high blood pressure or high cholesterol.

The present invention includes methods or bispecific antibodies (for use), medicaments or pharmaceutical formulations comprising administering a therapeutically effective amount of a bispecific anti-VEGF/ANG2 antibody (or a medicament or pharmaceutical formulation comprising the bispecific anti-VEGF/ANG2 antibody) to a subject in need thereof.

In certain embodiments of the methods or bispecific antibodies (for use), medicaments or pharmaceutical formulations of the disclosure,

In certain embodiments, the bispecific antibody, medicament or pharmaceutical formulation comprising such bispecific anti-VEGF/ANG2 antibody is administered (intravitreally) to the subject in multiple doses, e.g., as part of a specific therapeutic dosing regimen.

In certain other embodiments, the dosing interval is extended if ERM is not present.

In certain embodiments of the methods or bispecific antibodies (for use), medicaments or pharmaceutical formulations of the disclosure, the dosing interval is shortened or maintained if ERM is present.

In certain embodiments of the methods or bispecific antibodies (for use), medicaments or pharmaceutical formulations of the disclosure, the patient does not have ERM prior to the treatment with the bispecific antibody. In certain embodiments of the methods or bispecific antibodies (for use), medicaments or pharmaceutical formulations of the disclosure, the administration prolongs the time to loss of visual acuity (e.g., reduces the progression and/or severity of the disease).

In one embodiment of the methods or bispecific antibodies (for use), medicaments or pharmaceutical formulations of the disclosure, the bispecific antibody is administered in a dose of about 5 to 7 mg (at each treatment). In one embodiment the bispecific antibody is administered in a dose of 6 mg+/−10% (at each treatment). In one embodiment the bispecific antibody is administered in a dose of about 6 mg (at each treatment).

In certain embodiments of the methods or bispecific antibodies (for use), medicaments or pharmaceutical formulations of the disclosure, the bispecific antibody is administered every 8 weeks or less frequently (e.g., every 2 months). For example, the bispecific antibody is administered every 9 weeks or less frequently, every 10 weeks or less frequently, every 11 weeks or less frequently, every 12 weeks or less frequently (e.g., every 3 months), every 13 weeks or less frequently, every 14 weeks or less frequently, every 15 weeks or less frequently, every 16 weeks or less frequently (e.g., every 4 months).

In certain embodiments, the bispecific antibody is administered every 8 to 10 weeks, every 10 to 12 weeks, every 11 to 13 weeks, every 12 to 14 weeks, every 13 to 15 weeks, or every 14 to 16 weeks.

Such method, use, bispecific antibody (for use), medicament or pharmaceutical formulation may comprise sequentially administering initial doses (“treatment initiation”) (e.g. 3 to 7 monthly administrations; in one embodiment the treatment initiation includes 3 to 4 monthly administrations, in one embodiment the treatment initiation includes 4 to 5 monthly administrations; in one embodiment the treatment initiation includes 4 to 6 monthly administrations; in one embodiment the treatment initiation includes at least 4 monthly administrations; in one embodiment the treatment initiation includes 5 to 7 monthly administrations, in one embodiment the treatment initiation includes 6 monthly administrations) followed by one or more secondary doses of a therapeutically effective amount of the bispecific antibody, medicament or pharmaceutical formulation.

In certain embodiments of the methods or bispecific antibodies (for use), medicaments or pharmaceutical formulations of the disclosure, the bispecific antibody is administered following a treatment initiation, wherein the treatment initiation comprises 3 to 7 monthly (e.g., every 4 weeks) administrations.

In one embodiment, the bispecific antibody, medicament or pharmaceutical formulation is administered every 10 to 12 weeks (following treatment initiation). In one embodiment, the bispecific antibody, medicament or pharmaceutical formulation is administered every 11 to 13 weeks (following treatment initiation). In one embodiment, the bispecific antibody, medicament or pharmaceutical formulation is administered every 12 to 14 weeks (following treatment initiation). In one embodiment, the bispecific antibody, medicament or pharmaceutical formulation is administered every 13 to 15 weeks (following treatment initiation). In one embodiment, the bispecific antibody, medicament or pharmaceutical formulation is administered every 14 to 16 weeks (following treatment initiation).

In certain embodiments of the methods or bispecific antibodies (for use), medicaments or pharmaceutical formulations of the disclosure, the bispecific antibody is administered at a concentration of about 110 to 130 mg/mL. In certain embodiments, the bispecific antibody is administered at a concentration of about 120 mg/mL.

The bispecific antibody of the disclosure may be administered in a liquid pharmaceutical formulation. Suitable liquid formulation is disclosed in International Patent Application Publication No. WO2020/089051, which is incorporated herein in its entirety.

For example, in certain embodiments, the liquid pharmaceutical formulation comprises:
about 110 to 130 mg/mL of the bispecific antibody comprising,
about 15 to 35 mM of sodium, and
about 15 to 25 mM of a histidine acetate buffer,

and having a pH of 5.5±0.5.

In certain embodiments, the liquid pharmaceutical formulation further comprises one or more of:
about 7.0 mM±2.0 mM methionine;
about 0.03% to 0.07% (w/v) polysorbate 20; and
about 160 mM±24 mM sucrose.

Such liquid pharmaceutical formulation, in certain embodiments, has a viscosity of about 20 mPas or less, and/or a turbidity of about 30 FTU or less, and/or an ionic strength between about 20 and 50. International Patent Application Publication No. WO2020/089051, which is incorporated herein in its entirety, describes suitable methods to determine viscosity, turbidity and ionic strength. In certain embodiments, the liquid pharmaceutical formulation is essentially free of visible particles. In certain other embodiments, the liquid pharmaceutical formulation is essentially free of (or does not comprise) calcium chloride and/or arginine.

Antibody specificity refers to selective recognition of the antibody for a particular epitope of an antigen. Natural antibodies, for example, are monospecific.

“Bispecific antibodies” according to the invention are antibodies which have two different antigen-binding specificities. Antibodies of the present invention are specific for two different antigens, VEGF as first antigen and ANG-2 as second antigen.

The term “monospecific” antibody as used herein denotes an antibody that has one or more binding sites each of which bind to the same epitope of the same antigen.

The term “valent” as used within the current application denotes the presence of a specified number of binding sites in an antibody molecule. As such, the terms “bivalent”, “tetravalent”, and “hexavalent” denote the presence of two binding site, four binding sites, and six binding sites, respectively, in an antibody molecule. The bispecific antibodies according to the invention are preferably “bivalent”.

The terms “bispecific antibody which binds to human vascular endothelial growth factor (VEGF) and to human angiopoietin-2 (ANG-2)”, “bispecific anti-VEGF/ANG2 antibody” and “bispecific <VEGF/ANG2> antibody” as used herein are interchangeable and refer to an antibody which has at least two different antigen-binding sites, a first one which binds to VEGF and a second one which binds to ANG2.

Bispecific anti-VEGF/ANG2 antibodies are e.g. described in International Patent Application Publication Nos. WO2010/040508, WO2011/117329, WO2012/131078, WO2015/083978, WO2017/197199, and WO2014/009465. WO2014/009465 describes bispecific anti-VEGF/ANG2 antibodies especially designed for treatment of ocular vascular diseases. The bispecific anti-VEGF/ANG2 antibodies of WO2014/009465 (which is incorporated herein in its entirety) are especially useful in the treatment and treatment schedules of ocular vascular diseases as described herein. In particular, anti-VEGF/ANG2 antibody CrossMAb VEGFang2-0016 as described in WO2014/009465 which is also described as faricimab (in World Health Organization (2017). “International Nonproprietary Names for Pharmaceutical Substances (INN). Proposed INN: List 118” WHO Drug Information. 31 (4)) is a preferred bispecific anti-VEGF/ANG2 antibody of the present invention.

In one embodiment the bispecific antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat).

In one embodiment such bispecific anti-VEGF/ANG2 antibody is bivalent.

In one embodiment such bispecific, bivalent anti-VEGF/ANG2 antibody is characterized in that
i) said first antigen-binding site specifically binding to VEGF comprises as heavy chain variable domain VH an amino acid sequence of SEQ ID NO: 7, and as light chain variable domain VL an amino acid sequence of SEQ ID NO: 8, and
ii) said second antigen-binding site specifically binding to ANG-2 comprises as heavy chain variable domain VH an amino acid sequence of SEQ ID NO: 15, and as light chain variable domain VL an amino acid sequence of SEQ ID NO: 16.

In one aspect of the invention such bispecific, bivalent antibody according to the invention is characterized in comprising
a) the heavy chain and the light chain of a first full length antibody that specifically binds to VEGF;
b) the modified heavy chain and modified light chain of a second full length antibody that specifically binds to ANG-2, wherein the constant domains CL and CH1 are replaced by each other.

This bispecific, bivalent antibody format for the bispecific antibody specifically binding to human vascular endothelial growth factor (VEGF) and human angiopoietin-2 (ANG-2) is described in WO 2009/080253 (including Knobs-into-Holes modified CH3 domains). The antibodies based on this bispecific, bivalent antibody format are named CrossMAbs.

In one embodiment such bispecific, bivalent anti-VEGF/ANG2 antibody is characterized in comprising:
a) as heavy chain of the first full length antibody the amino acid sequence of SEQ ID NO: 17, and as light chain of the first full length antibody the amino acid sequence of SEQ ID NO: 18, and
b) as modified heavy chain of the second full length antibody the amino acid sequence of SEQ ID NO: 19, and as modified light chain of the second full length antibody the amino acid sequence of SEQ ID NO: 20.

In one embodiment such bispecific, bivalent anti-VEGF/ANG2 antibody is characterized in comprising the amino acid sequences of SEQ ID NO: 17, of SEQ ID NO: 18, of SEQ ID NO: 19, and of SEQ ID NO: 20.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one embodiment the CH3 domains of the bispecific, bivalent antibody according to the invention is altered by the “knob-into-holes” technology which is described in detail with several examples in e.g. WO 96/027011, Ridgway J. B., et al., Protein Eng 9 (1996) 617-621; and Merchant, A. M., et al., Nat Biotechnol 16 (1998) 677-681. In this method the interaction surfaces of the two CH3 domains are altered to increase the heterodimerization of both heavy chains containing these two CH3 domains. Each of the two CH3 domains (of the two heavy chains) can be the “knob”, while the other is the “hole”. The introduction of a disulfide bridge stabilizes the heterodimers (Merchant, A. M, et al., Nature Biotech 16 (1998) 677-681; Atwell, S., et al. J. Mol. Biol. 270 (1997) 26-35) and increases the yield.

In a preferred aspect of the invention the bispecific anti-VEGF/ANG2 antibodies according to the invention are characterized in that
the CH3 domain of one heavy chain and the CH3 domain of the other heavy chain each meet at an interface which comprises an original interface between the antibody CH3 domains;
wherein said interface is altered to promote the formation of the bispecific antibody, wherein the alteration is characterized in that:
a) the CH3 domain of one heavy chain is altered,
so that within the original interface the CH3 domain of one heavy chain that meets the original interface of the CH3 domain of the other heavy chain within the bispecific antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and
b) the CH3 domain of the other heavy chain is altered,
so that within the original interface of the second CH3 domain that meets the original interface of the first CH3 domain within the bispecific antibody
an amino acid residue is replaced with an amino acid residue having a smaller side chain volume, thereby generating a cavity within the interface of the second CH3 domain within which a protuberance within the interface of the first CH3 domain is positionable.

Thus the bispecific anti-VEGF/ANG2 antibodies for use described herein are preferably characterized in that
the CH3 domain of the heavy chain of the full length antibody of a) and the CH3 domain of the heavy chain of the full length antibody of b) each meet at an interface which comprises an alteration in the original interface between the antibody CH3 domains;
wherein i) in the CH3 domain of one heavy chain
an amino acid residue is replaced with an amino acid residue having a larger side chain volume, thereby generating a protuberance within the interface of the CH3 domain of one heavy chain which is positionable in a cavity within the interface of the CH3 domain of the other heavy chain
and wherein
ii) in the CH3 domain of the other heavy chain an amino acid residue is replaced with an amino acid residue having a smaller side chain volume, thereby generating a cavity within the interface of the second CH3 domain within which a protuberance within the interface of the first CH3 domain is positionable.

Preferably said amino acid residue having a larger side chain volume is selected from the group consisting of arginine (R), phenylalanine (F), tyrosine (Y), tryptophan (W).

Preferably said amino acid residue having a smaller side chain volume is selected from the group consisting of alanine (A), serine (S), threonine (T), valine (V).

In one aspect of the invention both CH3 domains are further altered by the introduction of cysteine (C) as amino acid in the corresponding positions of each CH3 domain such that a disulfide bridge between both CH3 domains can be formed.

In one embodiment, the bispecific antibody comprises a T366W mutation in the CH3 domain of the “knobs chain” and T366S, L368A, Y407V mutations in the CH3 domain of the “hole chain”. An additional interchain disulfide bridge between the CH3 domains can also be used (Merchant, A. M, et al., Nature Biotech 16 (1998) 677-681) e.g. by introducing a S354C mutation into one CH3 domain and a Y349C mutation into the other CH3 domain.

In a another preferred embodiment the bispecific antibody comprises S354C and T366W mutations in one of the two CH3 domains and Y349C, T366S, L368A, Y407V mutations in the other of the two CH3 domains In a another preferred embodiment the bispecific antibody comprises Y349C, T366W mutations in one of the two CH3 domains and S354C, T366S, L368A, Y407V mutations in the other of the two CH3 domains (the additional Y349C or S354C mutation in one CH3 domain and the additional S354C or Y349C mutation in the other CH3 domain forming a interchain disulfide bridge) (numbering always according to EU index of Kabat (Kabat, E. A., et al., Sequences of Proteins of Immunological Interest, 5th ed., Public Health Service, National Institutes of Health, Bethesda, MD (1991)).

Other techniques for CH3-modifications to enforce the heterodimerization are contemplated as alternatives of the invention and described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291.

In one embodiment the heterodimerization approach described in EP 1 870 459A1 is used alternatively. This approach is based on the introduction of substitutions/mutations of charged amino acids with the opposite charge at specific amino acid positions of the in the CH3/CH3 domain interface between both heavy chains. One preferred embodiment for said multispecific antibodies are amino acid R409D and K370E mutations in the CH3 domain of one heavy chain and amino acid D399K and E357K mutations in the CH3 domain of the other heavy chain of the multispecific antibody (numberings according to Kabat EU index).

In another embodiment said multispecific antibody comprises an amino acid T366W mutation in the CH3 domain of the “knobs chain” and amino acid T366S, L368A and Y407V mutations in the CH3 domain of the “hole chain”; and additionally comprises amino acid R409D and K370E mutations in the CH3 domain of the “knobs chain” and amino acid D399K and E357K mutations in the CH3 domain of the “hole chain”.

In one embodiment the heterodimerization approach described in WO2013/157953 is used alternatively. In one embodiment the CH3 domain of one heavy chain comprises an amino acid T366K mutation and the CH3 domain of the other heavy chain comprises an amino acid L351D mutation. In a further embodiment the CH3 domain of the one heavy chain further comprises an amino acid L351K mutation. In a further embodiment the CH3 domain of the other heavy chain further comprises an amino acid mutation selected from Y349E, Y349D and L368E (in one embodiment L368E).

In one embodiment the heterodimerization approach described in WO2012/058768 is used alternatively. In one embodiment the CH3 domain of one heavy chain comprises amino acid L351Y and Y407A mutations and the CH3 domain of the other heavy chain comprises amino acid T366A and K409F mutations. In a further embodiment the CH3 domain of the other heavy chain further comprises an amino acid mutation at position T411, D399, S400, F405, N390 or K392. In one embodiment said amino acid mutation is selected from the group consisting of
a) T411N, T411R, T411Q, T411K, T411D, T411E and T411W,
b) D399R, D399W, D399Y and D399K,
c) S400E, S400D, S400R and S400K,
d) F405I, F405M, F405T, F405S, F405V and F405W,
e) N390R, N390K and N390D,
f) K392V, K392M, K392R, K392L, K392F and K392E.

In a further embodiment the CH3 domain of one heavy chain comprises amino acid L351Y and Y407A mutations and the CH3 domain of the other heavy chain comprises amino acid T366V and K409F mutations. In a further embodiment the CH3 domain of one heavy chain comprises an amino acid Y407A mutation and the CH3 domain of the other heavy chain comprises amino acid T366A and K409F mutations. In a further embodiment the CH3 domain of the other heavy chain further comprises amino acid K392E, T411E, D399R and S400R mutations.

In one embodiment the heterodimerization approach described in WO2011/143545 is used alternatively. In one embodiment the amino acid modification according to WO2011/143545 is introduced in the CH3 domain of the heavy chain at a position selected from the group consisting of 368 and 409.

In one embodiment the heterodimerization approach described in WO2011/090762 which also uses the knob-into-hole technology described above is used alternatively. In one embodiment the CH3 domain of one heavy chain comprises an amino acid T366W mutation and the CH3 domain of the other heavy chain comprises an amino acid Y407A mutation. In one embodiment the CH3 domain of one heavy chain comprises an amino acid T366Y mutation and the CH3 domain of the other heavy chain comprises an amino acid Y407T mutation.

In one embodiment the multispecific antibody is of IgG2 isotype and the heterodimerization approach described in WO2010/129304 is used alternatively.

In one embodiment the heterodimerization approach described in WO2009/089004 is used alternatively. In one embodiment the CH3 domain of one heavy chain comprises an amino acid substitution of K392 or N392 with a negatively-charged amino acid (in one embodiment glutamic acid (E) or aspartic acid (D); in a further embodiment a K392D or N392D mutation) and the CH3 domain of the other heavy chain comprises an amino acid substitution of D399, E356, D356, or E357 with a positively-charged amino acid (in one embodiment Lysine (K) or arginine (R), in a further embodiment a D399K, E356K, D356K or E357K substitution; and in an even further embodiment a D399K or E356K mutation). In a further embodiment the CH3 domain of the one heavy chain further comprises an amino acid substitution of K409 or R409 with a negatively-charged amino acid (in one embodiment glutamic acid (E) or aspartic acid (D); in a further embodiment a K409D or R409D mutation). In a further embodiment the CH3 domain of the one heavy chain further or alternatively comprises an amino acid substitution of K439 and/or K370 with a negatively-charged amino acid (in one embodiment glutamic acid (E) or aspartic acid (D)).

In one embodiment the heterodimerization approach described in WO2007/147901 is used alternatively. In one embodiment the CH3 domain of one heavy chain comprises amino acid K253E, D282K and K322D mutations and the CH3 domain of the other heavy chain comprises amino acid D239K, E240K and K292D mutations.

In one embodiment the heterodimerization approach described in WO2007/110205 is used alternatively.

In one preferred embodiment such bispecific anti-VEGF/ANG2 antibody is bivalent.

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a T366W mutation is comprised in one CH3 domain and T366S, L368A, Y407V mutations are comprised the other CH3 domain (numberings according to EU Index of Kabat).

In one embodiment the bispecific, bivalent antibody which binds to human vascular endothelial growth factor (VEGF) and to human angiopoietin-2 (ANG-2) is a bispecific anti-VEGF/ANG2 antibody comprising a first antigen-binding site that specifically binds to human VEGF and a second antigen-binding site that specifically binds to human ANG-2, wherein
i) said first antigen-binding site specifically binding to VEGF comprises in the heavy chain variable domain a CDR3H region of SEQ ID NO: 1, a CDR2H region of SEQ ID NO: 2, and a CDR1H region of SEQ ID NO:3, and in the light chain variable domain a CDR3L region of SEQ ID NO: 4, a CDR2L region of SEQ ID NO:5, and a CDR1L region of SEQ ID NO:6; and
ii) said second antigen-binding site specifically binding to ANG-2 comprises in the heavy chain variable domain a CDR3H region of SEQ ID NO: 9, a CDR2H region of, SEQ ID NO: 10, and a CDR1H region of SEQ ID NO: 11, and in the light chain variable domain a CDR3L region of SEQ ID NO: 12, a CDR2L region of SEQ ID NO: 13, and a CDR1L region of SEQ ID NO: 14, and wherein
iii) the bispecific antibody comprises a constant heavy chain region of human IgG1 subclass comprising the mutations I253A, H310A, and H435A and the mutations L234A, L235A and P329G (numberings according to EU Index of Kabat; and wherein
iv) in the constant heavy chain region a S354C and T366W mutations are comprised in one CH3 domain and Y349C, T366S, L368A and Y407V mutations are comprised the other CH3 domain (numberings according to EU Index of Kabat).

In one embodiment such bispecific, bivalent anti-VEGF/ANG2 is characterized in comprising the amino acid sequences of SEQ ID NO: 17, of SEQ ID NO: 18, of SEQ ID NO: 19, and of SEQ ID NO: 20.

Accordingly, one embodiment of the invention is a bispecific, bivalent antibody comprising a first antigen-binding site that specifically binds to human VEGF and a second antigen-binding site that specifically binds to human ANG-2, characterized in comprising the amino acid sequences of SEQ ID NO: 17, of SEQ ID NO: 18, of SEQ ID NO: 19, and of SEQ ID NO: 20.

In one preferred embodiment such bispecific anti-VEGF/ANG2 antibody is faricimab.

The term “VEGF” as used herein refers to human vascular endothelial growth factor (VEGF/VEGF-A), the 165-amino acid human vascular endothelial cell growth factor (amino acid 27-191 of precursor sequence of human VEGF165: SEQ ID NO: 25; amino acids 1-26 represent the signal peptide), and related 121, 189, and 206 vascular endothelial cell growth factor isoforms, as described by Leung, D. W., et al., Science 246 (1989) 1306-9; Houck et al., Mol. Endocrin. 5 (1991) 1806-1814; Keck, P. J., et al., Science 246 (1989) 1309-12 and Connolly, D. T., et al., J. Biol. Chem. 264 (1989) 20017-24; together with the naturally occurring allelic and processed forms of those growth factors. VEGF is involved in the regulation of normal and abnormal angiogenesis and neovascularization associated with tumors and intraocular disorders (Ferrara, N., et al., Endocr. Rev. 18 (1997) 4-25; Berkman, R. A., et al., J. Clin. Invest. 91 (1993) 153-159; Brown, L. F., et al., Human Pathol. 26 (1995) 86-91; Brown, L. F., et al., Cancer Res. 53 (1993) 4727-4735; Mattem, J., et al., Brit. J. Cancer. 73 (1996) 931-934; and Dvorak, H. F., et al., Am. J. Pathol. 146 (1995) 1029-1039). VEGF is a homodimeric glycoprotein that has been isolated from several sources and includes several isoforms. VEGF shows highly specific mitogenic activity for endothelial cells. A VEGF antagonist/inhibitor inhibits binding of VEGF to its receptor VEGFR. Known VEGF antagonist/inhibitors include bispecific anti-VEGF/ANG2 antibodies as described in WO2014/009465.

The term “ANG-2” as used herein refers to human angiopoietin-2 (ANG-2) (alternatively abbreviated with ANGPT2 or ANG2) (SEQ ID NO: 24) which is described e.g. in Maisonpierre, P. C., et al, Science 277 (1997) 55-60 and Cheung, A. H., et al., Genomics 48 (1998) 389-91. The angiopoietins-1 and -2 were discovered as ligands for the Ties, a family of tyrosine kinases that is selectively expressed within the vascular endothelium (Yancopoulos, G. D., et al., Nature 407 (2000) 242-48). There are now four definitive members of the angiopoietin family. Angiopoietin-3 and -4 (Ang-3 and Ang-4) may represent widely diverged counterparts of the same gene locus in mouse and man (Kim, I., et al., FEBS Let, 443 (1999) 353-56; Kim, I., et al., J Biol Chem 274 (1999) 26523-28). ANG-1 and ANG-2 were originally identified in tissue culture experiments as agonist and antagonist, respectively (see for ANG-1: Davis, S., et al., Cell 87 (1996) 1161-69; and for ANG-2: Maisonpierre, P. C., et al., Science 277 (1997) 55-60). All of the known angiopoietins bind primarily to its receptor TIE2, and both Ang-1 and -2 bind to TIE2 with an affinity of 3 nM (Kd) (Maisonpierre, P. C., et al., Science 277 (1997) 55-60). An ANG2 antagonist/inhibitor inhibits binding of ANG2 to its receptor TIE2. Known ANG2 antagonist/inhibitors include bispecific anti-VEGF/ANG2 antibodies as described in WO2014/009465.

An antigen-binding sites of the bispecific antibody of the invention contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences.

The antibodies of the invention comprise immunoglobulin constant regions derived from human origin of immunoglobulin class IgG1.

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are of particular interest. Other forms of “chimeric antibodies” encompassed by the present invention are those in which the constant region has been modified or changed from that of the original antibody to generate the desired properties according to the invention, especially in regard to C1q binding and/or Fc receptor (FcR) binding. Such chimeric antibodies are also referred to as “class-switched antibodies”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See e.g. Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric antibodies. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boemer, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to C1q binding and/or FcR binding, e.g. by “class switching” i.e. change or mutation of Fc parts (e.g. from IgG1 to IgG4 and/or IgG1/IgG4 mutation).

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies according to the invention have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The “variable region” (variable region of a light chain (VL), variable region of a heavy chain (VH)) or “variable domain” as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention. The term “antigen-binding portion of an antibody” when used herein refers to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, MD (1991) and/or those residues from a “hypervariable loop”.

The term “epitope” includes any polypeptide determinant capable of specific binding to an antibody. In certain embodiments, epitope determinant includes chemically active surface groupings of molecules such as amino acids, sugar side chains, phosphoryl, or sulfonyl, and, in certain embodiments, may have specific three-dimensional structural characteristics, and or specific charge characteristics. An epitope is a region of an antigen that is bound by an antibody.

The term “full length antibody” denotes an antibody consisting of two “full length antibody heavy chains” and two “full length antibody light chains”. A “full length antibody heavy chain” is a polypeptide consisting in N-terminal to C-terminal direction of an antibody heavy chain variable domain (VH), an antibody constant heavy chain domain 1 (CHI), an antibody hinge region (HR), an antibody heavy chain constant domain 2 (CH2), and an antibody heavy chain constant domain 3 (CH3), abbreviated as VH-CHT-HR-CH2-CH3; and optionally an antibody heavy chain constant domain 4 (CH4) in case of an antibody of the subclass IgE. Preferably the “full length antibody heavy chain” is a polypeptide consisting in N-terminal to C-terminal direction of VH, CHI, HR, CH2 and CH3. A “full length antibody light chain” is a polypeptide consisting in N-terminal to C-terminal direction of an antibody light chain variable domain (VL), and an antibody light chain constant domain (CL), abbreviated as VL-CL. The antibody light chain constant domain (CL) can be kappa or lambda. The two full length antibody chains are linked together via inter-polypeptide disulfide bonds between the CL domain and the CHI domain and between the hinge regions of the full length antibody heavy chains. Examples of typical full length antibodies are natural antibodies like IgG (e.g. IgG1 and IgG2), IgM, IgA, IgD, and IgE. The full length antibodies according to the invention can be from a single species e.g. human, or they can be chimerized or humanized antibodies. The full length antibodies according to the invention comprise two antigen binding sites each formed by a pair of VH and VL, which both specifically bind to the same antigen. The C-terminus of the heavy or light chain of said full length antibody denotes the last amino acid at the C-terminus of said heavy or light chain. The N-terminus of the heavy or light chain of said full length antibody denotes the last amino acid at the N-terminus of said heavy or light chain.

The term “constant region” or “constant domains” as used within the current applications denotes the sum of the domains of an antibody other than the variable region. The constant region is not involved directly in binding of an antigen, but exhibits various effector functions. Depending on the amino acid sequence of the constant region of their heavy chains, antibodies are divided in the classes: IgA, IgD, IgE, IgG and IgM, and several of these may be further divided into subclasses, such as IgG1, IgG2, IgG3, and IgG4, IgA1 and IgA2. The heavy chain constant regions that correspond to the different classes of antibodies are called alpha, delta., epsilon., gamma, and micro, respectively. The light chain constant regions which can be found in all five antibody classes are called kappa and lambda.

The term “constant region derived from human origin” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., (see e.g. Johnson, G., and Wu, T. T., Nucleic Acids Res. 28 (2000) 214-218; Kabat, E. A., et al, Proc. Natl. Acad. Sci. USA 72 (1975) 2785-2788).

The term constant heavy chain domain (or region) as used herein defines a C-terminal region of an immunoglobulin heavy chain that contains at least a portion of the constant heavy chain region.

The term includes native sequences of the constant heavy chain domains and variant constant heavy chain domains. Variant constant heavy chain domains include e.g. mutations in the constant domain which are used to foster the heterodimerization as described above for the knobsinto hole technology. Also, other mutations like e.g. L234A (Leu235Ala), L235A (Leu234Ala) and P329G (Pro329Gly) can be included as constant domains with such mutations have a reduced FcR binding (especially they show no more binding to FcRgammaI, FcRgammaII and FcRgammaIII). This is especially useful to reduce potential side effects like e.g. thrombosis (Meyer, T., et al., J. Thromb. Haemost. 7 (2009) 171-81). In addition, e.g. also the mutations I253A, H310A, and H435A (numbering according to EU Index of Kabat) can be included in the constant domain as constant domains with such mutations have a reduced FcRn one or two mutations) or eliminated FcRn binding (all 3 mutations).

In one aspect, a human IgG heavy chain constant region extends from alanine118 (A118) (numbering according to EU index of Kabat) to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the constant heavy chain domain may or may not be present. Amino acid sequences of heavy chains including the constant heavy chain domain are denoted herein with C-terminal glycine-lysine dipeptide if not indicated otherwise.

In one embodiment the bispecific antibodies according to the invention have a constant region of human IgG1 subclass (derived from human IgG1 subclass). However, the C-terminal lysine (Lys447), or the C-terminal glycine (Gly446) and the C-terminal lysine (Lys447), of the Fe region may or may not be present.

In one embodiment the bispecific antibody as described herein is of IgG1 isotype/subclass and comprises a constant heavy chain domain of SEQ ID NO: 23 or the constant parts of the heavy chain amino acid sequence of SEQ ID NO: 17 and of the heavy chain amino acid sequence of SEQ ID NO: 18. In one embodiment additionally the C-terminal glycine (Gly446) is present. In one embodiment additionally the C-terminal glycine (Gly446) and the C-terminal lysine (Lys447) is present.

Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

In one embodiment the bispecific antibody according to the invention is of human IgG1 subclass with mutations L234A (Leu235Ala), L235A (Leu234Ala) and P329G (Pro329Gly). Such antibody has a reduced FcR binding (especially they show no more binding to FcRgammaI, FcRgammaII and FcRgammaIII). This is especially useful to reduce potential side effects like e.g. thrombosis (Meyer, T., et al., J. Thromb. Haemost. 7 (2009) 171-81).

While Pro329Ala mutation which was described already removes only two third of the FcgammaRIIIa sandwich interaction, the Pro329Gly in the antibodies according to the invention fully imparts binding of the Fc part to FcgammaRIII. This is especially useful as the binding to FcgammaRIII is involved in ADCC (antibody-dependent cellular toxicity) which leads to cell death, which may be helpful in the treatment of cancer diseases, but which can cause serious side effect in the antibody based treatment of other vascular or immunological diseases. So the antibodies according to the invention of IgG1 subclass with mutations L234A, L235A and P329G and IgG4 subclass with mutations S228P, L235E and P329G are especially useful, as they both show no more binding to FcRgammaI, FcRgammaII and FcRgammaIlI.

An “effective amount” of an agent, e.g., a pharmaceutical formulation or bispecific anti-VEGF/ANG2 antibody, refers to an amount effective, at dosages and for periods of time necessary, to achieve the desired therapeutic or prophylactic result.

In one embodiment of the invention the bispecific antibody, medicament or pharmaceutical formulation as described herein is administered via intravitreal application, e.g. via intravitreal injection (is administered “intravitreally”). This can be performed in accordance with standard procedures known in the art. See, e.g., Ritter et al., J. Clin. Invest. 116 (2006) 3266-76; Russelakis-Carneiro et al., Neuropathol. Appl. Neurobiol. 25 (1999) 196-206; and Wray et al., Arch. Neurol. 33 (1976) 183-5.

In some embodiments, therapeutic kits of the invention can contain one or more doses of the bispecific antibody described present in a medicament or pharmaceutical formulation, a suitable device for intravitreal injection of the medicament or pharmaceutical formulation, and an instruction detailing suitable subjects and protocols for carrying out the injection. In these embodiments, the medicament or pharmaceutical formulation are typically administered to the subject in need of treatment via intravitreal injection. This can be performed in accordance with standard procedures known in the art. See, e.g., Ritter et al., J. Clin. Invest. 116 (2006) 3266-76; Russelakis-Carneiro et al., Neuropathol. Appl. Neurobiol. 25 (1999) 196-206; and Wray et al., Arch. Neurol. 33 (1976) 183-5.

Regardless of the route of administration selected, the bispecific antibody as described herein is formulated into pharmaceutically acceptable dosage forms by conventional methods known to those of skill in the art.

The compositions and methods of the disclosure are illustrated further by the following example, which is not to be construed as limiting the disclosure in scope or spirit to the specific methods and compositions described in them.

### Example

### Evaluation of Epiretinal Membrane Formation in Diabetic Macular Edema (DME)

Faricimab is a bispecific anti-Ang-2 and anti-VEGF-A antibody evaluated in the phase 3 YOSEMITE (ClinicalTrials.gov identifier: NCT03622580) and RHINE (ClinicalTrials.gov identifier: NCT03622593) trials in eyes with DME (Wykoff C C, et al. Efficacy, durability, and safety of intravitreal faricimab with extended dosing up to every 16 weeks in patients with diabetic macular oedema (YOSEMITE and RHINE): two randomized, double-masked, phase 3 trials. The Lancet. 2022; 399(10326):741-755; Eter N, et al. YOSEMITE and RHINE: Phase 3 Randomized Clinical Trials of Faricimab for Diabetic Macular Edema: Study Design and Rationale. Ophthalmol Sci. 2022; 2(1):100111). Eligible eyes (N=1891) received 1 of 3 treatment regimens for 2 years: intravitreal faricimab every 8 weeks (Q8W), faricimab Q8W according to a personalized treat-and-extend-based regimen (T&E) with dosing extended up to every 16 weeks (Q16W) based on central subfield thickness (CST) and best-corrected visual acuity (BCVA) criteria, or intravitreal aflibercept Q8W. A major exclusion criteria for the trials was the presence of ERMs at baseline. Consequently, the trial population was used to establish whether dual inhibition of Ang-2 and VEGF-A with faricimab decreases ERM formation compared to VEGF inhibition with aflibercept.

The primary objective of this study was to compare the 2-year incidence of ERM formation in eyes with DME treated with faricimab Q8W, faricimab T&E, or aflibercept Q8W. A secondary objective was to conduct a retrospective analysis of baseline factors that may be associated with the development of ERMs among eyes with DME receiving intravitreal injections. Tertiary objectives were to describe the functional and anatomical impact of ERMs in eyes with DME analyzed in a treatment-agnostic manner and to understand the potential impact of ERMs on treatment burden.

### Materials and Methods:

The YOSEMITE and RHINE trials were 2 identically designed, double-masked, multicenter, randomized, parallel-group, registrational phase 3 studies of faricimab in patients with DME. The studies were designed to evaluate the efficacy, safety, pharmacokinetics, and durability of intravitreal faricimab 6.0 mg for the treatment of DME when dosed either every 8 weeks or according to a PTI regimen in adjustable intervals (up to every 16 weeks), compared with intravitreal aflibercept 2.0 mg dosed every 8 weeks as per the label.

The YOSEMITE and RHINE trials enrolled 1891 patients (YOSEMITE, 940 patients; RHINE, 951 patients). The studies comprised 3 treatment arms: (1) faricimab 6.0 mg monthly (every 4 weeks, Q4W) for 6 months followed by every-8-week dosing (Q8W); (2) faricimab 6.0 mg every 4 weeks for 4 months followed by per PTI, a protocol-driven T&E regimen with up to every-16-week dosing; or (3) aflibercept 2.0 mg every 4 weeks (Q4W) for 5 months followed by every-8-week dosing (Q8W), in line with the product label. Patients were randomized 1:1:1 to each of the 3 treatment arms of the studies. Randomization was stratified by baseline best-corrected visual acuity (BCVA) Early Treatment Diabetic Retinopathy Study (ETDRS) letter score (64 ETDRS letters or better vs. 63 letters or worse; Snellen equivalent threshold, ˜20/63), prior intravitreal anti-VEGF therapy (yes vs. no), and region (United States and Canada, Asia, and the rest of the world). The goal of stratification was to prevent imbalance of these potentially confounding variables across the study arms that could affect the interpretation of study outcomes. To preserve masking, patients were seen every 4 weeks and underwent a sham procedure at study treatment visits when they were not treated with active study drug. The study included a screening period of up to 28 days, a 96-week treatment period, and a final study visit at week 100 (year 2).

### Study Participants and Eligibility Criteria

Patients 18 years of age or older with center-involving DME secondary to type 1 or type 2 diabetes mellitus were eligible to participate. The inclusion criterion for hemoglobin Alc level was set at up to 10% to limit enrollment of patients with unstable diabetic control to minimize any potential changes to the outcome variables that could be secondary to large fluctuations in underlying glucose levels. General exclusion criteria included, among others, untreated diabetes or treatment initiated within 3 months of day 1; uncontrolled high blood pressure; and history of other disease, physical examination finding, or clinical laboratory finding suggestive of a condition that would contraindicate use of any of the study drugs, may affect interpretation of the study results, or in the opinion of the investigator would render the patient at high risk for treatment complications.

One eye per patient was designated as the study eye. A major exclusion criterion was the presence of an ERM involving the fovea or disrupting the macular architecture in the study eye, as evaluated by a central reading center (CRC; Duke Reading Center, Durham, NC or Vienna Reading Center, Vienna, Austria). Further ocular exclusion and inclusion criteria for the study eye are shown in Table 1. The central reading centers (CRCs) evaluated the spectral-domain (SD) OCT and color fundus photography (CFP) images obtained at screening to provide an objective, masked assessment of whether patients' study eyes met the study eligibility criteria. If both eyes were eligible for inclusion, the eye with the worse BCVA at screening was selected as the study eye.

TABLE 1
Ocular Exclusion and Inclusion Criteria for the Study Eye

| Exclusion Criteria | Inclusion Criteria |
|---|---|
| High-risk PDR in the study eye (any vitreous or | Macular thickening secondary to DME |
| preretinal hemorrhage; neovascularization | involving the center of the fovea, with CST |
| elsewhere one-half disc area or more within an | ≥325 μm (defined as the thickness from the |
| area equivalent to the mydriatic ETDRS 7 fields | ILM to Bruch's membrane), measured by |
| on clinical examination or on CFP images; | SD OCT or SS OCT (Spectralis |
| neovascularization at disc one-third disc area or | [Heidelberg Engineering GmbH, |
| more on clinical examination), as graded by the | Heidelberg, Germany], Topcon [Topcon, |
| CRCs | Tokyo, Japan], or Cirrus [Carl Zeiss |
| Tractional retinal detachment, preretinal | Meditec, Dublin, CA]) in the central 1-mm |
| fibrosis, or epiretinal membrane involving the | area of the macula as graded by the CRCs |
| fovea or disrupting the macular architecture in | BCVA between 25 and 73 ETDRS letters |
| the study eye | (approximate Snellen equivalent, 20/320- |
| Active rubeosis | 20/40), as assessed on the standardized |
| Uncontrolled glaucoma | ETDRS chart at 4 mos |
| History of retinal detachment or macular hole | Sufficiently clear ocular media and |
| (stage 3 or 4) | adequate pupillary dilatation to allow |
| Aphakia or implantation of anterior chamber | acquisition of good-quality CFP images |
| intraocular lens | (including ETDRS 7 modified fields or 4 |
| Intravitreal anti-VEGF treatment within 3 | wide-angle fields to permit grading of DR |
| months^{§} (previously treated patients) or any | and assessment of the retina) and other |
| intravitreal anti-VEGF agents in study eye | imaging methods |
| before day 1 (treatment-naïve patients) | |
| Treatment with PRP within 3 mos^{§} | |
| Macular (focal or grid) laser within 3 mos^{§} | |
| Any cataract surgery or treatment for | |
| complications of cataract surgery with steroids | |
| or YAG laser capsulotomy within 3 mos^{§} | |
| Any other intraocular surgery | |
| Any intravitreal or periocular (sub-Tenon) | |
| corticosteroid treatment within 6 mos^{§} | |
| Any use of medicated intraocular implants, | |
| including Ozurdex (Allergan USA, Inc., | |
| Madison, NJ), within 6 mos^{§} | |
| Any use of Iluvien implants at any time | |
| Treatment for other retinal diseases that can | |
| lead to macular edema | |

PRP: panretinal photocoagulation; SD: spectral-domain; SS: swept-source; YAG: yttrium-aluminum-garnet.
^{§}Before day 1 of study.

Study eyes were permitted to be either anti-VEGF treatment naïve (with no previous history of intravitreal anti-VEGF therapy) or previously anti-VEGF treated (provided that the last treatment was ≥3 months before the day 1 study visit). Study eyes previously treated with anti-VEGF therapy were capped at 25% of the total patient enrollment for each study. The rationale for capping the number of patients previously treated with anti-VEGF therapy was based on the heterogeneous nature of this patient population, with a potential history of long-standing and potentially insufficiently treated DME, resulting in pharmacologically irreversible macular damage that could thus limit the possibility of visual acuity improvements.

### Assessments

ERMs were prospectively evaluated at baseline, and at weeks 16, 48, 52, 56, 92, 96, and 100 by CRC assessors who were masked to treatment assignment. An ERM was defined as a membrane on the internal limiting membrane (ILM) causing significant distortion of the macular architecture in the central 1-mm subfield visualized using optical coherence tomography (OCT). At the beginning of the trials, the two reading centers harmonized their interpretation of ERMs with deformation and showed a high rate of agreement (κ=0.46-0.77; data not shown).

BCVA and CST (defined as the distance between the ILM and Bruch's membrane in the central 1-mm diameter of the Early Treatment Diabetic Retinopathy [ETDRS] grid) were assessed every 4 weeks through week 100. The proportion of eyes in the faricimab T&E group receiving treatment Q4W, Q8W, every 12 weeks (Q12W), and Q16W was evaluated through week 96.

### Statistical Analysis

Efficacy analyses were performed on eyes that did not have ERMs at baseline and had baseline/post-baseline ERM assessments, grouped according to treatment at randomization. All p-values are nominal and no formal conclusions should be made based on p-values. A descriptive summary of baseline characteristics was provided to evaluate the balance among treatment groups. The cumulative incidence of ERM formation over 2 years was calculated using the proportions of eyes that developed an ERM over 100 weeks. The denominator is the number of eyes without ERMs at baseline who had ERM status available through the study. The numerator is the number of eyes with ERMs based on the assumption that ERMs remain present throughout the trial once detected. Logistic regression analysis was used to assess the effect of treatment on the risk of developing ERMs after adjusting for stratification factors at randomization. Odds ratios (OR) and the corresponding 95% confidence intervals (CI) were estimated for each of the faricimab treatment arms vs. aflibercept.

A retrospective analysis was conducted to explore the relationship between ocular and socio-demographic factors and ERM development. Separate logistic regression analyses were initially conducted on a predefined set of variables along with the treatment group. Variables with a p-value <0.05 were subsequently included in a multivariable logistic regression model. The final multivariable model included sex, age, lens status, baseline BCVA, baseline macular leakage (mm²), and treatment group as covariates. Adjusted ORs and 95% CI were calculated to determine the potential factors associated with the development of ERMs.

Descriptive analyses were conducted to compare BCVA, CST, macular leakage area, and the proportions of eyes with intraretinal fluid (IRF) or subretinal fluid (SRF) on OCT scans at week 96 or 100 in eyes that did and did not develop ERMs throughout the study. Data were analyzed in a treatment-agnostic manner due to the small number of eyes that developed ERMs and the associated difficulty conducting meaningful comparisons across treatment arms.

To understand the potential impact of ERMs on treatment durability, descriptive analyses were conducted to evaluate the treatment intervals at week 96 in eyes that did and did not develop ERMs in the faricimab T&E arm. Specifically, the proportion of eyes on Q4W, Q8W, Q12W, and Q16W treatment intervals at week 96 were reported.

### Results

### Study Population.

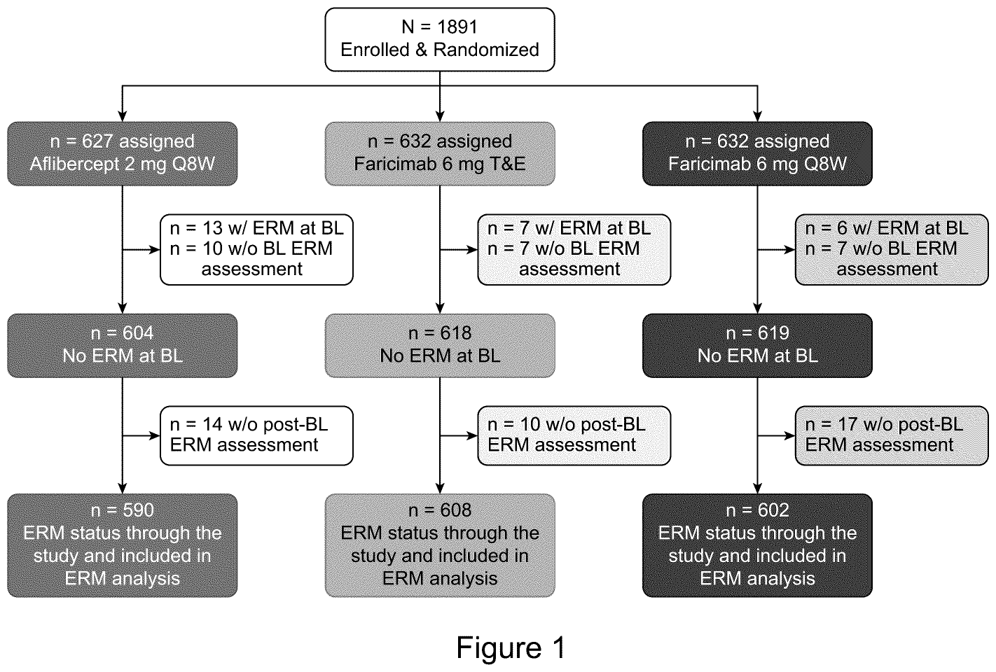

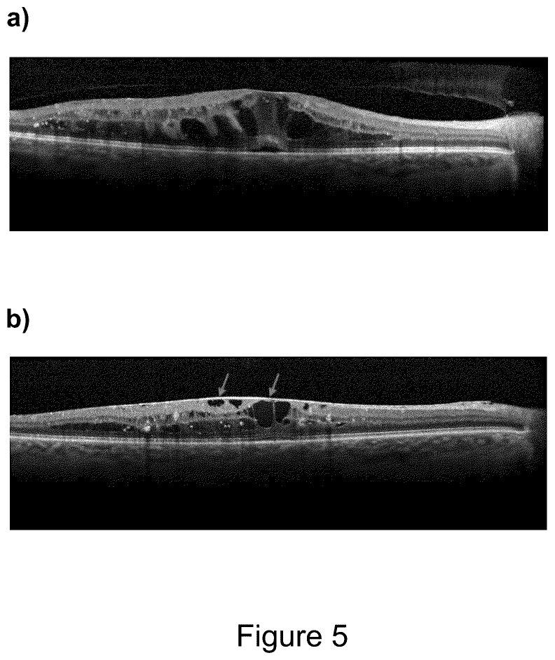

The YOSEMITE and RHINE trials enrolled 1,891 eyes with DME, 632, 632, and 627 of which were randomized to faricimab Q8W, faricimab T&E, and aflibercept Q8W groups, respectively. Eligibility for the trials (including ERM status) was assessed at screening, whilst ERM status at baseline may have been assessed by a different grader. Inherent variability in the Reader assessment of ERMs in borderline cases meant the study graders occasionally identified an ERM that was not called an ERM by the eligibility grader. This accounts for the small number of enrolled eyes with ERMs, all of which were excluded from the analysis. Overall, 619, 618, and 604 eyes in the faricimab Q8W, faricimab T&E, and aflibercept Q8W groups, respectively, had ERM assessments showing no ERMs at baseline and were included in the analysis (FIG. 1). An example of an OCT scan showing an eye without ERMs is shown in FIG. 5a. Baseline socio-demographic and ocular characteristics were generally balanced across treatment groups (Table 2).

TABLE 2
Baseline Socio-Demographic and Ocular Characteristics
Among Patients with No ERMs at Baseline.

| | Aflibercept | | Faricimab | | Faricimab | |
|---|---|---|---|---|---|---|
| Baseline socio-demographic and ocular | Q8W | | T&E | | Q8W | |
| characteristics | n = 604 | | n = 618 | | n = 619 | |
| Age, mean (SD) years | 62.3 | (9.7) | 62.2 | (10.0) | 62.0 | (9.8) |
| Sex, n (%) | | | | | | |
| Male | 349 | (57.8) | 385 | (62.3) | 374 | (60.4) |
| Female | 255 | (42.2) | 233 | (37.7) | 245 | (39.6) |
| Race, n (%) | | | | | | |
| White | 483 | (80.0) | 476 | (77.0) | 482 | (77.9) |
| Asian | 59 | (9.8) | 61 | (9.9) | 63 | (10.2) |
| Black or African American | 36 | (6.0) | 48 | (7.8) | 39 | (6.3) |
| American Indian or Alaska Native | 8 | (1.3) | 5 | (0.8) | 5 | (0.8) |
| Native Hawaiian or other Pacific Islander | 3 | (0.5) | 0 | | 4 | (0.6) |
| BMI, mean (SD) kg/m² | 30.6 | (6.3) | 30.4 | (6.4) | 30.7 | (6.5) |
| Smoking status, n (%) | | | | | | |
| Never smoked | 385 | (63.7) | 375 | (60.7) | 356 | (57.5) |
| Previous smoker | 168 | (27.8) | 184 | (29.8) | 195 | (31.5) |
| Current smoker | 51 | (8.4) | 59 | (9.5) | 68 | (11.0) |
| HbA_{1c}, mean (SD) | 7.6 | (1.1) | 7.7 | (1.1) | 7.6 | (1.1) |
| T2DM, n (%) | 575 | (95.2) | 586 | (94.8) | 577 | (93.2) |
| Time since DME diagnosis, mean (SD) months | 18.8 | (32.5) | 19.3 | (35.0) | 16.4 | (27.5) |
| BCVA | | | | | | |
| Mean (SD), ETDRS letters, | 62.2 | (9.4) | 62.2 | (9.8) | 62.1 | (9.9) |
| [Snellen equivalent] | [20/63] | | [20/63] | | [20/63] | |
| CST, mean (SD) μm | 478.7 | (129.2) | 477.8 | (129.1) | 477.4 | (126.9) |
| IOP, mean (SD) mmHg | 15.1 | (3.1) | 15.2 | (3.0) | 15.3 | (3.1) |
| Previously treated with anti-VEGF, n (%) | 133 | (22.0) | 130 | (21.0) | 136 | (22.0) |
| Severe NPDR or worse, n (%)^{a} | 134 | (22.2) | 132 | (21.4) | 135 | (21.8) |
| Phakic status, n (%) | | | | | | |
| Phakic | 448 | (74.2) | 461 | (74.6) | 467 | (75.4) |
| Pseudophakic | 151 | (25.0) | 153 | (24.8) | 149 | (24.1) |
| Other | 5 | (0.8) | 4 | (0.6) | 3 | (0.5) |
| Posterior vitreous detachment, n (%) | 109 | (18.0) | 113 | (18.3) | 101 | (16.3) |
| Macular Leakage^{b}, mean (SD) mm² | 23.0 | (12.4) | 23.1 | (12.3) | 23.2 | (12.3) |

^{a}Defined as DRS level ≥53 excluding level 90 (cannot grade).
^{b}Only 1736 of 1800 patients with ERM status post-Baseline were found to have a Baseline Macular Leakage measurement, 580 in the Faricimab Q8W arm, 591 in the Faricimab T&E arm, and 565 in the Aflibercept Q8W arm.
BMI, body mass index; HbA_{1c}, glycated hemoglobin; IOP, intraocular pressure; NPDR, non-proliferative diabetic retinopathy; SD, standard deviation; T2DM, type 2 diabetes mellitus; T&E, treat and extend-based regimen.

### ERM Development Across Intravitreal Therapies.

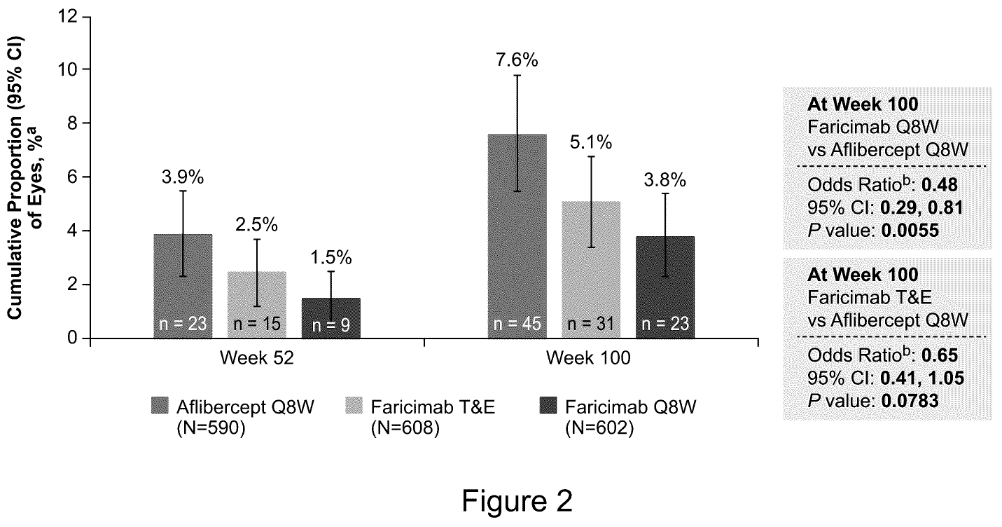

Of the 1,891 study eyes randomized to treatment, 91 either had ERMs at baseline or were not assessed for ERMs at or post-baseline. Thus, 602, 608 and 590 eyes in the faricimab Q8W, faricimab T&E, and aflibercept Q8W groups, respectively, were included in the analysis for incident ERMs (FIG. 1). The respective cumulative incidence of ERM formation in eyes that received faricimab Q8W, faricimab T&E, or aflibercept Q8W was 1.5% (9/602), 2.5% (15/608), and 3.9% (23/590) at 1 year, and 3.8% (23/602), 5.1% (31/608), and 7.6% (45/590) at 2 years. An example of an OCT scan showing ERM formation at Week 48 is shown in FIG. 5b. The odds of ERM formation over 2 years were reduced by 52% with faricimab Q8W vs. aflibercept Q8W (OR 0.48, 95% CI 0.29-0.81, p=0.0055; FIG. 2) with a trend towards reduced odds for faricimab T&E vs. aflibercept Q8W (OR 0.65, 95% CI 0.41-1.05, p=0.0783).

### ERM Development: Ocular and Socio-Demographic Risk Factors.

Retrospective univariate analysis of baseline factors associated with ERM development are described in Table 3. Eyes that developed ERMs had a poorer mean BCVA at baseline (58.8 ETDRS letters [20/80 Snellen] vs. 62.4 letters [20/63 Snellen]; p=0.0004), were less frequently phakic (63.6% vs 75.8%; p=0.0047), had greater baseline macular leakage area (27.5 vs 22.8 mm², p=0.0005), and were less likely to be treated with faricimab Q8W (23.3% vs 34.0%; p=0.0054) than eyes that did not develop ERMs. In addition, patients with eyes that developed ERMs were older (64.3 years vs. 62.0 years; p=0.0207) and were more likely to be female (46.5% vs. 39.2%; p=0.0499). According to univariate analysis, the presence of investigator-reported posterior vitreous detachment (PVD) was not associated with ERM development (p=0.6697). Based on multivariable logistic regression analysis, factors associated with ERM development were treatment with faricimab Q8W (OR 0.48, 95% CI 0.28-0.82), greater baseline macular leakage area (OR for a 10 mm² increase: 1.41, 95% CI 1.16-1.72), and (to a lesser extent) treatment with faricimab T&E (OR 0.67, 95% CI 0.41-1.10) (Table 4). Multivariable logistic regression analysis with stratification factors as covariates found that eyes developing ERMs were less likely to have baseline BCVA≥64 EDTRS letters (≥20/50 Snellen) vs. <64 letters (<20/50 Snellen) (OR 0.58, 95% CI 0.38-0.87) (Table 5).

TABLE 3
Retrospective Univariate Analysis of Baseline Demographic
and Ocular Characteristics (ERM Analysis Population^{a})

| | Participants | | Participants | | |
|---|---|---|---|---|---|
| | with ERM | | without ERM | | |
| Baseline socio-demographic | formation | | formation | | |
| and ocular characteristics | (n = 99) | | (n = 1701) | | P value |
| Age, mean (SD) years | 64.3 | (8.6) | 62.0 | (9.8) | 0.0207 |
| Sex, n (%) | | | | | |
| Male | 50 | (50.5) | 1035 | (60.8) | — |
| Female | 49 | (49.5) | 666 | (39.2) | 0.0499 |
| Race, n (%) | | | | | |
| White | 86 | (86.9) | 1324 | (77.8) | 0.1347 |
| Black or African American | 3 | (3.0) | 116 | (6.8) | 0.5530 |
| Other^{b} | 7 | (7.1) | 197 | (11.6) | — |
| BMI, mean (SD) kg/m² | 31.4 | (6.9) | 30.6 | (6.4) | 0.2063 |
| Smoking status, n (%) | | | | | |
| Never smoked | 66 | (66.7) | 1021 | (60.0) | 0.3143 |
| Previous smoker | 26 | (26.3) | 510 | (30.0) | 0.6429 |
| Current smoker | 7 | (7.1) | 170 | (10.0) | — |
| HbA_{1c}, mean (SD) % | 7.5 | (1.0) | 7.6 | (1.1) | 0.3453 |
| T1DM, n (%)^{c} | | | | | |
| Yes | 1 | (1.0) | 103 | (6.1) | 0.0739 |
| No/T2DM | 98 | (99.0) | 1598 | (93.9) | — |
| Time since DME diagnosis, | 17.2 | (24.9) | 18.3 | (32.3) | 0.7286 |
| mean (SD) months | | | | | |
| Mean BCVA (SD) ETDRS | 58.8 (11.2) | | 62.4 (9.5) | | 0.0004 |
| letters [Snellen equivalent] | [20/80] | | [20/63] | | |
| CST, mean (SD) μm | 492.8 | (140.1) | 476.9 | (127.5) | 0.2487 |
| IOP, mean (SD) mmHg | 14.7 | (2.9) | 15.2 | (3.1) | 0.1103 |
| Previously treated with anti- | 28 | (28.3) | 366 | (21.5) | 0.1131 |
| VEGF, n (%) | | | | | |
| Severe NPDR or worse, n | 26 | (26.2) | 368 | (21.7) | 0.2961 |
| (%)^{d} | | | | | |
| Lens status, n (%) | | | | | |
| Phakic | 63 | (63.6) | 1290 | (75.8) | 0.0047 |
| Pseudophakic | 36 | (36.4) | 399 | (23.5) | — |
| Other | 0 | | 12 | (0.1) | 0.9867 |
| Posterior vitreal detachment, | 19 | (19.2) | 295 | (17.3) | 0.6697 |
| n (%) | | | | | |
| Type of treatment, n (%) | | | | | |
| Faricimab Q8W | 23 | (23.2) | 579 | (34.0) | 0.0054 |
| Faricimab T&E | 31 | (31.3) | 577 | (33.9) | 0.0745 |
| Aflibercept Q8W | 45 | (45.5) | 545 | (32.0) | — |
| Number of study drug | 14.1 | (3.39) | 13.1 | (3.03) | NA |
| administrations, mean (SD) | | | | | |
| Macular Leakage^{e}, mean | 27.5 | (11.6) | 22.8 | (12.3) | 0.0005 |

(SD) mm²

^{a}41 patients were removed from the analysis due to missing values for the response variable.
^{b}Asian, American Indian or Alaska Native, Native Hawaiian or other Pacific Islander, multiple and unknown races.
^{c}Reference for baseline T1DM included participants who had no T1DM and had T2DM: 3 patients who had both T1DM and T2DM at baseline were included as having T1DM at baseline.
^{d}Defined as DRS level ≥53 excluding level 90 (cannot grade).
^{e}Only 1736 of 1800 patients with ERM status post-baseline were found to have a baseline macular leakage measurement, 93 with ERM formation and 1643 without ERM formation. P values are from separate logistic regression models with each baseline factor and treatment group as covariates, except that the model on treatment is univariate.
DRS, diabetic retinopathy status; IOP, intraocular pressure; NA, not analyzed; NPDR, non-proliferative diabetic retinopathy; SD, standard deviation; T1DM, type 1 diabetes mellitus

TABLE 4
Multivariate Logistic Regression Analysis of Baseline Factors
Associated With ERM^{a} Formation (ERM Analysis Population)

| Baseline socio-demographic | | |
|---|---|---|
| and ocular characteristics | OR | 95% CI |
| Age | 1.02 | 1.00-1.05 |
| Sex | | |

| | |
|---|---|
| Male | Reference |

| | | |
|---|---|---|
| Female | 1.35 | 0.88-2.07 |
| Lens status | | |

| | |
|---|---|
| Pseudophakic | Reference |

| | | |
|---|---|---|
| Phakic | 0.67 | 0.42-1.09 |
| Type of treatment | | |

| | |
|---|---|
| Aflibercept Q8W | Reference |

| | | |
|---|---|---|
| Faricimab Q8W | 0.48 | 0.28-0.82 |
| Faricimab T&E | 0.67 | 0.41-1.10 |
| Baseline BCVA | 0.98 | 0.96-1.00 |
| Baseline Macular Leakageb | 1.41 | 1.16-1.72 |

^{a}Model n = 1733, 64 patients of the 1800 with post-baseline ERM status are missing baseline macular leakage, and 3 patients are missing Baseline BCVA.
^{b}The unit increase for this odds ratio is 10 mm², meaning a patient with 10 mm² greater macular leakage area in the study eye at Baseline had 41% greater odds of incurring ERM post-Baseline.

TABLE 5
Multivariate Logistic Regression Analysis of
Baseline Stratification Factors Associated
with ERM Formation (ERM Analysis Population)

| Baseline stratification factors | OR | 95% CI |
|---|---|---|
| BCVA | | |

| | |
|---|---|
| <64 EDTRS letters (<20/50 Snelllen) | Reference |

| | | |
|---|---|---|
| ≥64 EDTRS letters (≥20/50 Snellen) | 0.58 | 0.38-0.87 |
| Prior intravitreal anti-VEGF | | |

| | |
|---|---|
| No | Reference |

| | | |
|---|---|---|
| Yes | 1.25 | 0.79-1.98 |
| Region | | |

| | |
|---|---|
| US/Canada | Reference |

| | | |
|---|---|---|
| Rest of world | 0.79 | 0.52-1.19 |
| Study | | |

| | |
|---|---|
| RHINE | Reference |

| | | |
|---|---|---|
| YOSEMITE | 1.37 | 0.90-2.08 |
| Type of treatment | | |

| | |
|---|---|
| Aflibercept | Reference |

| | | |
|---|---|---|
| Faricimab Q8W | 0.48 | 0.29-0.81 |
| Faricimab T&E | 0.65 | 0.41-1.05 |

### Visual Acuity and Anatomical Outcomes

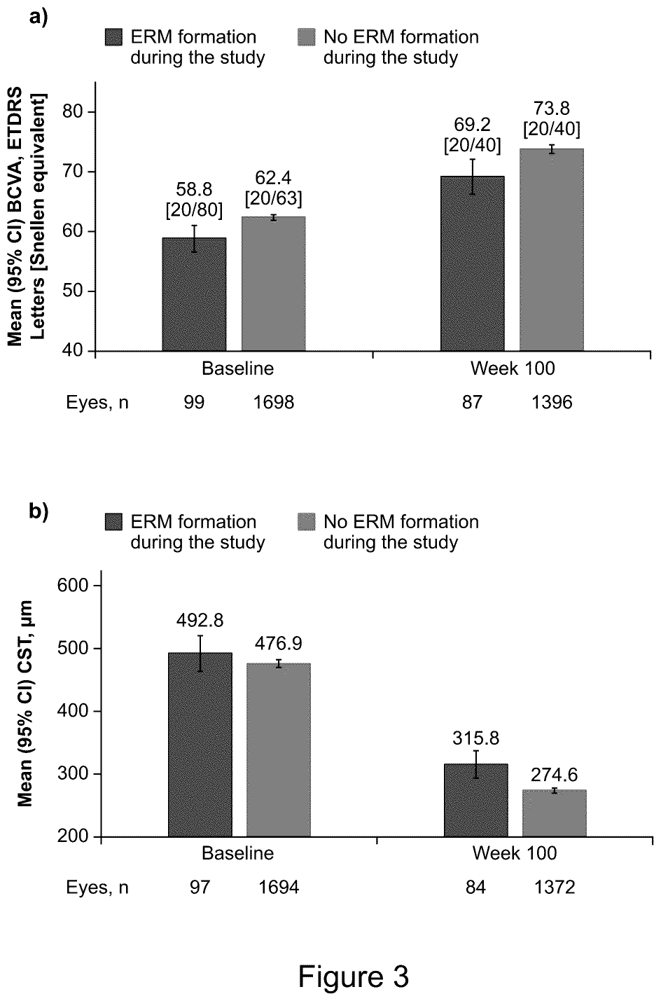

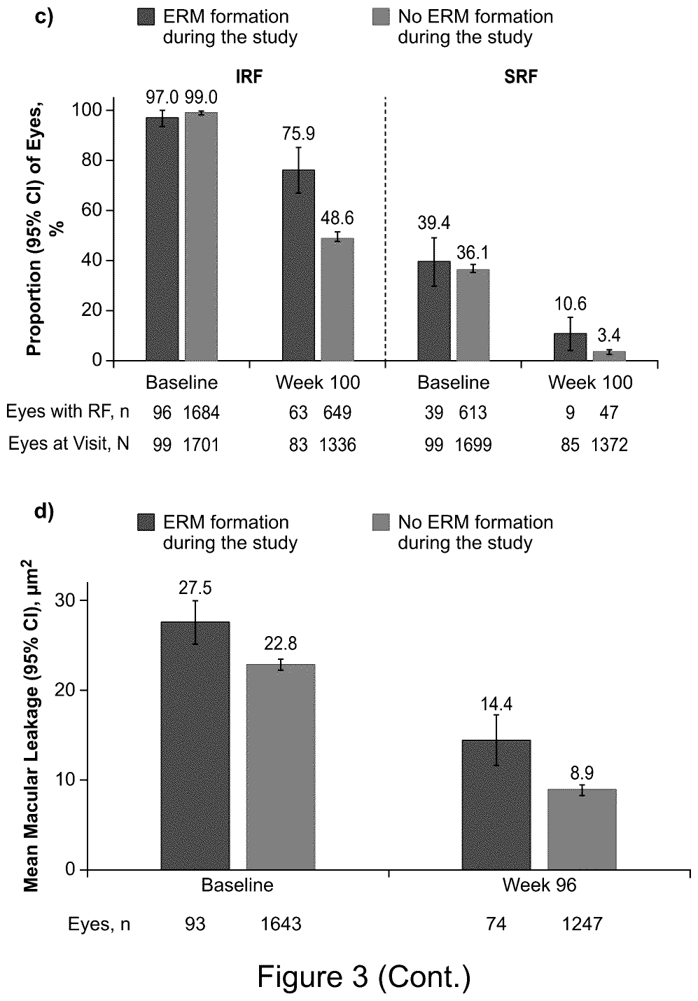

At 2 years (100 weeks), eyes that developed ERMs across all 3 treatment groups had poorer BCVA (mean (SD) 69.2 (13.6) ETDRS letters [20/40 Snellen] vs. 73.8 (13.1) letters [20/40 Snellen], respectively) and thicker retinas (mean CST (SD) 315.8 (99.2) vs 274.6 (74.1) μm; FIGS. 3a and 3b and Table 6) than eyes that did not develop ERMs. A higher proportion of eyes with vs. without ERMs had IRF (75.9% vs 48.6%) or SRF (10.6% vs 3.4%) at year 2 (FIG. 3c). Eyes that developed ERMs had greater macular leakage area than eyes that did not develop ERMs (14.4 vs 8.9 mm²) at year 2 (FIG. 3d).

TABLE 6

| | All eyes that | All eyes that did not |
|---|---|---|
| | developed an ERM, n = 99 | develop an ERM, n = 1701 |
| | BCVA, mean (SD) ETDRS letters/[Snellen equivalent] | |
| Baseline | 58.8 (11.2)/[20/80] | 62.4 (9.5)/[20/63] |
| Week 52 | 68.8 (13.0)/[20/50] | 73.4 (11.5)/[20/40] |
| Week 100 | 69.2 (13.6)/[20/40] | 73.8 (13.1)/[20/40] |
| | CST, mean (SD) um | |
| Baseline | 492.8 (140.1) | 476.9 (127.5) |
| Week 52 | 330.1 (114.5) | 291.4 (80.3) |
| Week 100 | 315.8 (99.2) | 274.6 (74.1) |

### Durability Outcomes

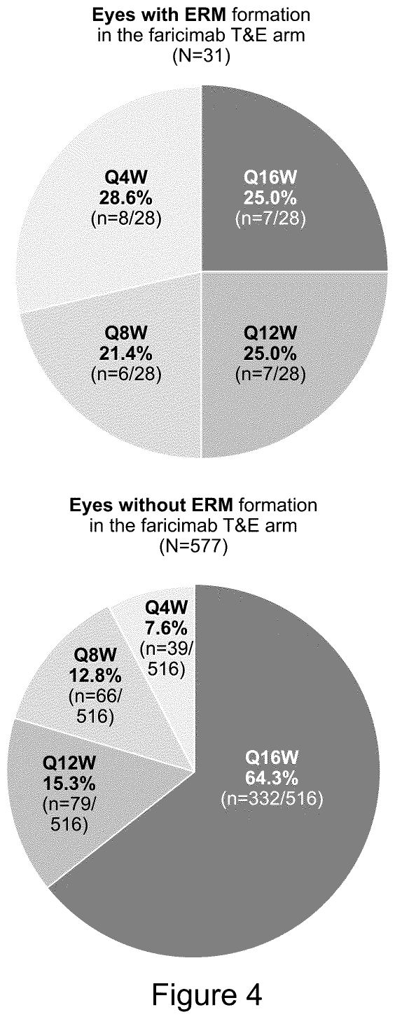

Of the 28 eyes treated with faricimab T&E that developed ERMs and completed the study, 14 (50.0%) achieved extended dosing (≥Q12W) at year 2 (96 weeks), with an equal number of eyes receiving treatment Q12W and Q16W (FIG. 4). In comparison, dosing in eyes treated with faricimab T&E that did not develop ERMs was extended in 411/516 eyes (79.7%), with 79/516 (15.3%) treated Q12W and 332/516 (63.3%) treated Q16W. Conversely, 8/28 (28.6%) vs. 39/516 (7.6%) eyes that did vs. did not develop ERMs were treated with faricimab Q4W at year 2, respectively.

### Discussion

In this analysis of phase 3 YOSEMITE/RHINE data in eyes with DME, treatment with faricimab Q8W reduced the risk of ERM formation by 52% vs. aflibercept Q8W. Previous studies show that the incidence of ERM formation after intravitreal injections in eyes with DME is about 9.5% over 2 years, while the incidence among the general population is approximately 4-5% over 5 years. In this study, the incidence of ERM formation over 2 years was 3.8%, 5.1%, and 7.6% following treatment with faricimab Q8W, faricimab T&E, or aflibercept Q8W, respectively.

Without being bound to a particular theory, it is believed that the comparatively favorable impact of faricimab vs. aflibercept on ERM development may be attributable to faricimab's anti-Ang-2 mechanism of action. Preclinical models of ischemic retinal diseases have demonstrated the potential of Ang-2 inhibition to reduce inflammation, stabilize the vasculature, and limit fibrosis. While the general etiology of ERM formation is multifactorial, there is strong evidence to suggest that ERMs are associated with inflammation and poor vasculature control. Hence, it is plausible from a biological perspective that the effect of faricimab on ERM formation is related to its anti-Ang-2 mechanism, thereby stabilizing vessels and reducing inflammation. Furthermore, the ability of faricimab to reduce IRF levels and CST thickness relative to aflibercept has been previously described, and this fluid control may contribute to the decreased formation of ERMs among patients treated with faricimab vs. aflibercept. Further research is required to understand the exact roles of the VEGF and Ang-2 pathways in ERM formation in patients with DME.

Retrospective multivariate logistic regression analysis showed that faricimab Q8W vs. aflibercept Q8W was associated with a reduced risk of ERM development (OR 0.48, 95% CI 0.28-0.82; p=0.0078) with a trend towards a reduced risk with faricimab T&E vs. aflibercept (OR 0.67, 95% CI 0.41-1.10; p=0.1129). Additionally, eyes with greater macular leakage at baseline had an increased risk of ERM development (OR for a 10 mm² increase in area: 1.41, 95% CI 1.16-1.72; p=0.0007). Such an association is consistent with prior literature citing macular leakage as a risk factor for ERMs. Multivariate logistic regression with stratification factors as covariates showed a BCVA≥64 (20/50 Snellen) vs. <63 ETDRS letters (20/63) was associated with an decreased risk of ERM formation (OR 0.58, 95% CI 0.38-0.87). The reason for this association is unknown. It is possible, however, that eyes with worse BCVA had more severe underlying disease, with greater inflammation and worse vascular stability.

Existing studies comprehensively describe the impact of ERMs on vision and anatomy, and the potential need for surgery. Yet there are limited data from large-scale phase 3 trials that assess the detrimental impact of ERMs on visual, anatomical, and durability outcomes. The present study found that eyes that developed ERMs following treatment with faricimab or aflibercept had numerically worse visual acuity and thicker retinas when compared to those that did not develop ERMs at 2 years. Furthermore, a higher proportion of eyes that developed ERMs versus those that did not develop ERMs had IRF (76% vs 49%) and SRF (11% vs 3%) at 2 years. IRF is a major negative predictive factor regarding retinal functional and therapeutic outcomes in eyes with neovascular age-related macular degeneration (nAMD). In DME, IRF does not respond as readily to anti-VEGF therapy as is seen in nAMD, and persistence of IRF is associated with degenerative alteration of the affected retina. Without being bound by theory, it is believed that the non-VEGF-related mechanism of faricimab may reduce those complications.

This analysis demonstrated it was possible to extend the dosing interval to Q16W in only 25% of eyes that developed an ERM in the faricimab T&E arm (n=31) compared with 64% who did not develop an ERM (n=577). Without being bound by theory, it is believed that the ERMs prevented resolution of IRF, through reduced antibody permeability causing relative anti-VEGF resistance. Alternatively, the ERMs could have prevented fluid resolution, or the development of degenerative cavitations by a VEGF-independent mechanism. Regardless, persistent IRF, decreased visual acuity and shorter treatment intervals were all associated with ERMs. In certain embodiments, early treatment with faricimab could help improve overall patient outcomes and treatment burden among patients with DME.

A strength of this analysis is the generalizability of the results since the data are from the largest phase 3 trials in eyes with DME to date. Additionally, the analyzed population had no ERMs at baseline, allowing for a robust evaluation of the impact of various intravitreal therapies and dosing regimens on ERM development over 2 years. ERMs were also prospectively evaluated by two CRCs, which were masked to treatment assignment and harmonized on ERM staging prior to study initiation. Study limitations included failure to characterize ERMs by size and objective quantification. However, the ERM definition was standardized in that the membrane needed to have had an impact on macular architecture per OCT scan.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of preventing or reducing epiretinal membrane (ERM) formation in an eye of a patient suffering from Diabetic Macular Edema (DME), the method comprising:
administering to the patient an effective amount of a bispecific antibody which binds to human vascular endothelial growth factor (VEGF) and to human angiopoietin-2 (ANG-2), and comprises the amino acid sequences of SEQ ID NO: 17, of SEQ ID NO: 18, of SEQ ID NO: 19, and of SEQ ID NO: 20.

2. The method of claim 1, wherein the effective amount of the bispecific antibody is sufficient to reduce ERM formation after 48 weeks of treatment.

3. The method of claim 1, wherein the effective amount of the bispecific antibody is sufficient to reduce ERM formation after 96 weeks of treatment.

4. The method of claim 3, wherein the ERM formation, if present, is less than 0.5 relative to the ERM formation with standard of care treatment (such as aflibercept).

5. The method of claim 3, wherein the ERM formation, if present, is less than 0.4 relative to the ERM formation without any treatment.

6. The method of claim 1, wherein the patient does not have ERM prior to the treatment with the bispecific antibody.

7. The method of claim 1, wherein preventing or reducing ERM formation prolongs the time to retreatment and/or prolongs the time to loss of visual acuity (e.g., reduces the progression and/or severity of the disease).

8. A method of treating a patient suffering from Diabetic Macular Edema (DME), the method comprising:
administering to the patient an effective amount of a bispecific antibody which binds to human vascular endothelial growth factor (VEGF) and to human angiopoietin-2 (ANG-2), and comprises the amino acid sequences of SEQ ID NO: 17, of SEQ ID NO: 18, of SEQ ID NO: 19, and of SEQ ID NO: 20;
measuring epiretinal membrane (ERM) in an eye of the patient after 16 and/or 48 weeks of treatment; and
adjusting administration dosing interval based on the ERM presence.

9. The method of claim 8, wherein the dosing interval is extended if ERM is not present.

10. The method of claim 8, wherein the dosing interval is maintained or shortened if ERM is present.

11. The method of claim 10, wherein the patient does not have ERM prior to the treatment with the bispecific antibody.

12. The method of claim 8, wherein the administration prolongs the time to loss of visual acuity (e.g., reduces the progression and/or severity of the disease).

13. The method of claim 1, wherein the bispecific antibody is faricimab.

14. The method of claim 1, wherein the bispecific antibody is administered in a dose of about 5 to 7 mg.

15. The method of claim 1, wherein the bispecific antibody is administered in a dose of about 6 mg.

16. The method of claim 1, wherein the bispecific antibody is administered every 8 weeks or less frequently.

17. The method of claim 1, wherein the bispecific antibody is administered every 12 weeks or less frequently.

18. The method of claim 1, wherein the bispecific antibody is administered every 16 weeks or less frequently.

19. The method of claim 16, wherein the bispecific antibody is administered following a treatment initiation, wherein the treatment initiation comprises 3 to 7 monthly (e.g., every 4 weeks) administrations.

20. The method of claim 1, wherein the bispecific antibody is administered at a concentration of about 120 mg/mL.

21. The method of claim 1, wherein the bispecific antibody is administered in a liquid pharmaceutical formulation comprising:
about 110 to 130 mg/mL of the bispecific antibody comprising,
about 15 to 35 mM of sodium, and
about 15 to 25 mM of a histidine acetate buffer,
and having a pH of 5.5±0.5.

22. The method of claim 21, wherein the liquid pharmaceutical formulation further comprises one or more of:
about 7.0 mM±2.0 mM methionine;
about 0.03% to 0.07% (w/v) polysorbate 20; and
about 160 mM±24 mM sucrose.

23. The method of claim 22, wherein the liquid pharmaceutical formulation has a viscosity of about 20 mPas or less, and/or a turbidity of about 30 FTU or less, and/or an ionic strength between about 20 and 50, and/or essentially free of visible particles.

24. The method of claim 1, wherein the bispecific antibody is administered intravitreally.

25. The method of claim 1, wherein the bispecific antibody is administered using a prefilled syringe.

26. The method of claim 1, wherein the patient has center-involving DME.


SEQUENCE LISTING (25 sequences)

SEQ ID NO: 1 - protein, 14 aa, synthetic construct
YPYYYGTSHWYFDV

SEQ ID NO: 2 - protein, 17 aa, synthetic construct
WINTYTGEPTYAADFKR

SEQ ID NO: 3 - protein, 5 aa, synthetic construct
HYGMN

SEQ ID NO: 4 - protein, 9 aa, synthetic construct
QQYSTVPWT

SEQ ID NO: 5 - protein, 7 aa, synthetic construct
FTSSLHS

SEQ ID NO: 6 - protein, 11 aa, synthetic construct
SASQDISNYLN

SEQ ID NO: 7 - protein, 123 aa, synthetic construct [sequence omitted]

SEQ ID NO: 8 - protein, 107 aa, synthetic construct [sequence omitted]

SEQ ID NO: 9 - protein, 20 aa, synthetic construct
SPNPYYYDSSGYYYPGAFDI

SEQ ID NO: 10 - protein, 17 aa, synthetic construct
WINPNSGGTNYAQKFQG

SEQ ID NO: 11 - protein, 5 aa, synthetic construct
GYYMH

SEQ ID NO: 12 - protein, 11 aa, synthetic construct
QVWDSSSDHWV

SEQ ID NO: 13 - protein, 7 aa, synthetic construct
DDSDRPS

SEQ ID NO: 14 - protein, 11 aa, synthetic construct
GGNNIGSKSVH

SEQ ID NO: 15 - protein, 129 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSS

SEQ ID NO: 16 - protein, 110 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSS

SEQ ID NO: 17 - protein, 453 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNAYTQKSLSLSPGK

SEQ ID NO: 18 - protein, 463 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMASRTPEVTCVVVDVSHEDPEVKFNWYVDGVEVHNAKTKPREEQYNSTYRVVSVLTVLAQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNAYTQKSLSLSPGK

SEQ ID NO: 19 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 213 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 21 - protein, 107 aa, Homo sapiens [sequence omitted]

SEQ ID NO: 22 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 330 aa, Homo sapiens [sequence omitted]

SEQ ID NO: 24 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 25 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF